(19) United States
(12) Patent Application Publication
(10) Pub. No.: US 2014/0228247 A1
(43) Pub. Date: Aug. 14, 2014

(54) SEQUENCE-SPECIFIC ANALYSIS OF NUCLEIC ACIDS

(71) Applicant: Universitat Rostock, Rostock (DE)

(72) Inventors: Gerd-Uwe Flechsig, Teterow (DE); Maren Mix, Rostock (DE); Simone Krüger, Rostock (DE)

(73) Assignee: Universitat Rostock, Rostock (DE)

(21) Appl. No.: 14/348,190

(22) PCT Filed: Sep. 25, 2012
(86) PCT No.: PCT/EP2012/068818

§ 371 Date: Mar. 28, 2014

(30) Foreign Application Priority Data
Sep. 28, 2011 (DE) 10 2011 114 984.1

(51) Int. Cl. C12Q 1/68

(52) U.S. Cl. CPC C12Q 1/6825; C12Q 1/6837
USPC 506/9; 435/6.11; 435/287.2; 506/16

(57) ABSTRACT

The invention relates to a method for the sequence-specific analysis of nucleic acids in a sample in which the nucleic acid is present at least partially as a double strand, which method uses electrochemical detection methods (e.g. with [OsO4(bipy)]). In particular, the methods comprise steps in which the at least partially double-stranded nucleic acid strands are converted by thermal denaturation to single strands which are termed target strands, and at least one nucleic acid strand designated protective strand is added, which protective strand can hybridize with a target strand, in order to form partial double-stranded segments, wherein the protective strands are shorter than the target strands, wherein the temperature of the sample is rapidly lowered to a temperature of below 5° C., preferably below 0° C. The invention also relates to devices which are suitable for these methods comprising a flow system in which the steps of the method can take place consecutively, having heatable sections for thermal denaturation as well as coolable sections for rapid cooling of the sample.

The invention concerns a method for the sequence-specific analysis of nucleic acids in a sample, in which the nucleic acid is at least partially present as a double strand, using electrochemical detection methods (e.g. with [OsO₄(bidy)]). In particular, the methods comprise converting the at least partially double stranded nucleic acid strands into single strands termed target strands by thermal denaturation, and adding at least one nucleic acid strand termed protective strand, which is able to hybridise with a target strand to form partially double stranded segments, wherein the protective strands are shorter than the target strands, wherein the temperature of the sample is rapidly lowered to a temperature of less than 0° C. The invention additionally concerns devices that are suitable for these methods comprising a flow system with heatable sections for thermal denaturation as well as coolable sections for rapid cooling of the sample in which the steps of the methods can take place consecutively.

### State of the Art

To facilitate an electrochemical detection of hybridization events of nucleic acids, molecules active in redox can be employed which are either covalently bound to the target or the reporter stand, bind themselves electrostatically to the phosphate groups of the nucleic acids or insert themselves into the double stand as intercalators. The covaletly bound, so-called redox markers are bound during labeling or manufacturing of the target or reporter strands. Alternatively, osmium(VIII)oxide may be used in the form of complexes with bipyridine. These complex compounds react specifically with the pyrimidine bases thymine, uracile and to a considerably lesser extend with cytosine. This reaction does not take place in an intact double strand. On the other hand, single strands wherein the pyrimidine bases have reacted with osmium complexes are unable to form double strands. These correlations have been analyzed by Palecek, Jelen and Fojta since the beginning of the 1980s and used in form of numerous examples for the detection of the nucleic acids (M. Foita et al. J. Am. Chem. Soc. 126 (2004) 6532; P. Kostecka et al. Bioelectrochemistry 63 (2004) 245; M. Foita et al. Electroanalysis 15 (2003) 431).

The modification of DNA with osmium (VIII) complexes can easily take place by addition of the osmium reagent to the analyte solution. After the modification the excess reagent is removed via dialysis in common, simple vessels for dialysis. Furthermore, the hybridization of target and probe is usually carried out on different surfaces. Especially suitable are so-called magnetic beads that carry immobilized probe strands on their surface and can be separated with a magnetic field from the analyte solution (and thereby from non-complementary strands and reagents used for analysis). According to this principle, any single stranded nucleic acid strand can be labeled. However, in the majority of the samples the nucleic acids are present in double stranded form. This applies for DNA as well as for RNA; which is in large parts double stranded due to secondary structures.

It exists the problem, that on the one hand nucleic acid single strands that are modified with osmium are no longer able to form double strands and that on the other hand intact double strands do not react with the osmium tetroxide complexes such as the [OsO₄(bipy)]. Different ways to solve this problem have been proposed in the art.

This can be solved by blocking the part of the single strand which shall hybridize with a probe at a later stage with a protective strand. This way, PCR products or native nucleic acid samples are successfully labeled with osmium without losing the ability for hybridization. The large number of osmium moieties that are bound to the target strands results in a very high sensitivity in the subsequent electrochemical analysis (WO 07/020093). Therein, e.g., double strands amplified in a PCR were denaturated thermally (95° C.), the protective strand was added in excess and cooled to 35° C. It is taught that the optimal temperature for hybridization is below the melting temperature and can be determined by means of a melting curve analysis.

The first great advantage of labelling nucleic acids with the help of osmium tetroxide complexes resides in the simple implementation. One merely has to supplement the DNA/RNA containing solution with the complex, e.g. [OsO₄(bipy)], and this compound reacts then spontaneously with all thymine bases in the solution that are not present in DNA double strands. Also other pyrimidine bases are attacked in this manner. However, cytosine reacts about 10 times slower than thymine. Uracile replaces the thymine in the RNA. It differs from thymine only by a missing methyl group and reacts slightly slower with osmium tetroxide bipyridine.

The second great advantage is the high sensitivity that is achieved when the target strand contains many thymine or uracile bases, respectively. This is because the largest part thereof will always appear as a single strand and therefore be attacked by the [OsO₄(bipy)]. On average, 50 to 200 thymine bases per strand can be expected in a PCR product. RNA can contain more than 500 uracile bases.

Flechsig et al., Anal. Chem. 2007, demonstrate that a hybridization of single strands with protective strands occurs optimally at 40° C. Reske et al., Talanta 2007, analyzed double stranded PCR products after generation of single strands via treatment with lambda exonuclease, before hybridization of the single strands with short protective strands at room temperature (RT) for 2 h and subsequent reaction with [OsO₄(bipy)] for 2 h at RT.

Mix et al., Electroanalysis 2009, describe the manufacture of single stranded DNA strands by asymmetrical PCR, also followed by hybridization of the singe strands with short protective strands at room temperature (RT) for 2 h and subsequent reaction with [OsO₄(bipy)] for 2 h at RT.

It is therefore the object of the invention to provide a method and a device which facilitate a fast and simple detection of the nucleic acid sequences of interest with redox markers such as osmium tetroxide complexes if need be without prior PCR as well as to allow the electrochemical detection at a working electrode immediately after, wherein an asymmetrical PCR, exonuclease digestion or similar methods for the generation of single strands can be abstained from. This is an advantage in particular when the sequences of interest are at least partially present as double strands in the sample, e.g. in the presence of a large excess of unspecific DNA or RNA. Advantageously, double stranded DNA can often be amplified by a classical, symmetrical PCR before detection, which results in an improved yield compared with an asymmetrical PCR and thereby in an improved selectivity and sensitivity.

### Solution of the Problem

This problem is solved by the subject matter of the claims, in particular by a method for sequence-specific detection of nucleic acid in a sample in which the nucleic acid is at least partially present as a double strand, comprising
a) converting the at least partially double stranded nucleic acid strands into single strands termed target strands by thermal denaturation,
b) adding at least one nucleic acid strand termed protective strand, which is able to hybridize with a target strand to form partially double stranded segments, wherein the protective strands are shorter than the target strands, wherein the temperature of the sample is rapidly lowered to a temperature of less than 5 ° C., preferably less than 0° C.,
c) labeling the remaining single stranded segments of the target strands via reaction with a redox marker, which reacts selectively with the double bond of the pyrimidine rings of the nucleic acid strands and allows a electroanalytically usable redox reaction on working electrodes,
d) hybridizing the nucleic acid strands that are labeled in this manner on the surface of an electrode with probe strands that are immobilized thereon under replacement of the protective strands, and
e) detecting the nucleic acid strands that are hybridized to the probe strands electroanalytically.

The basic principle of the present invention resides in denaturing all nucleic acids (DNA and/or RNA) in a sample to single strands and blocking the unspecific segments (which are not of interest) with osmium tetroxide complexes, so that they are no longer able to hybridize. In contrast, the sequences of interest are protected in a manner known per se with short protective strands (see also DE 10 2005 039 726 or WO 07/020093).

Thereby, according to the invention the different speed of hybridisation of nucleic acid strands with a different length is exploited. This way, the long DNA and RNA strands will remain single strands for an extended period of time after e.g. thermal denaturation and chilling in an ice bath or freezing mixture while the short protective strands (typically 20 to 30 bases) can hybridize considerably faster. Due to thermal melting of the double stranded DNA and subsequent rapid cooling the long single strands cannot re-hybridize over an extended time period due to the kinetic inhibition. A comparison with Reske, Dissertation, 2009, shows that only very rapid cooling to low temperature in connection with according sufficiently long maintenance of the high and low temperature, respectively, is successful.

Within the scope of the invention protective strands which can form short double stranded segments are added during this phase. The segments protected in this manner will not be attacked during the subsequent modification with osmium tetroxide complexes and will be available for hybridization with capture probes later on. All remaining DNA or RNA segments are still present as single strands and will now be modified by means of osmium tetroxide complexes. These single stranded segments are now unable to hybridize.

In this manner, according to the invention, all unprotected DNA or RNA segments can be hidden when it subsequently comes to the hybridization with capture probes on gold electrodes. The unspecific attachment of non-complementary nucleic acid strands to the probe strands is made more difficult. The recognition of target strands in a surplus of non-complementary nucleic acid strands is, thus, made easier. According to the invention a selection of the nucleic acid sequence of interest is made twice: During the hybridization with the protective strands as well as upon the hybridization with the immobilized capture probes.

Preferably, the thermal denaturation according to step a) of the method of the invention takes place at at least 93° C., preferably at 95° C. or 97° C. for at least 5 min, preferably for at least 10 min. More preferably, a thermal denaturation takes place at 95° C. for at least 10 min.

According to the invention, the addition of the protective strand in step b) takes place at approximately the temperature of the thermal denaturation. In particular, it may take place at exactly this temperature or directly after the sample has been removed from an environment having this temperature. The temperature of the sample upon addition of the protective strands is also preferably approximately 80-95° C., more preferably approximately 90-95° C.

In one embodiment, the periods for denaturation of the double stranded segments of the DNA or RNA targets and for the hybridization with protective strands are longer than 1 min, preferably at least 5 min or at least 10 min.

Within the scope of the method according to the invention, the temperature of the sample is subsequently rapidly lowered to a temperature of less than 5° C., preferably 0° C. or less, more preferably to approximately −1° C. to −5° C., to −2° C. to −4° C., or to −2° C. to −3° C., in particular to about −2.5° C. by transferring the sample to an environment of 0° C. or less immediately after addition of the protective strand in step b). A rapid lowering of the temperature can e.g. be achieved by transferring the reaction vessel with the sample into an environment of 0° C. or less, e.g. into an ice bath. As the temperature of the sample is preferably lowered to less than 0° C., the ambient temperature in step b) is preferably approximately −5° C. to −25° C., preferably about −19° C. Such temperatures can, e.g. be reached in a freezing mixture of ice and a salt, wherein the salt is preferably NaCl and the salt solution is preferably saturated. Such a freezing mixture can e.g. have a temperature of −19° C. The rapid lowering of the temperature in step b) is accordingly achieved by a low ambient temperature (e.g. in a freezing mixture) and a good heat transmission (as e.g. in the freezing mixture).

Preferably, the temperature in step b) will be kept constant for at least 1 min, preferably at least 5 min or more preferably at least 10 min. Subsequently, a further hybridization of the protective strands at higher temperatures, in particular at RT (approx. 20-25° C.) is possible. This can happen for an additional 5-120 min, in particular 10-110 min or 30-60 min.

“Partially double stranded segments”, which are formed by protective strand and target strand in step b), is to be understood in the sense that the single stranded target strands hybridize only along a part of their entire length with protective strands and that, thereby, double stranded segments are formed, i.e. the length of the protective strands is chosen in a manner that sufficiently long single stranded segments of the target sequence are available for the subsequent reaction with the redox markers. These remaining single stranded segments of the target sequence have to contain at least one thymine base. The more thymine bases are present in the single stranded segment, the more can be labeled with the redox marker in the subsequent step and the higher the electroanalytical signals will be in the end, thereby increasing the sensitivity of the entire method.

The hybridization is carried out with protective strands that are shorter than the target strands. In particular, the protective strand is half as long as the target strand at most, ¼ as long at most or 1/10 as long at most. The protective strand preferably has a length of 10-200 b, more preferably of 15-100 b or 20-32 b. At the same time, the length of the protective strands has to be sufficient to enable the formation of a sufficiently strong double strand. On the other hand, a hybridization with probe strands, that are immobilized on the surface of an electrode is carried out in step d). As this hybridization occurs under replacement of the protective strands the following has to be considered: The probe strands should be precisely as long as the protective strands or only slightly longer. It is an advantage when mismatches occur in the target-protective strand-duplex. This advantage is particularly relevant when the probe strands are slightly shorter than the protective strands. The mismatches should not occur in positions of the target strands which carry thymine bases as these could otherwise be labeled which could influence a subsequent hybridization with the probe strands. This ensures that the target strand-protective strand-duplex is not more stable than the target strand-probe strand-duplex and that an exchange of the protective strands with the probe strands takes place.

Preferably, the protective strand is added in an excess of at least 2:1, preferably 3:1, 4:1, 5:1, 9:1 or 10:1 in step b).

Within the scope of the method according to the invention, the redox marker, which reacts selectively with the double bond of the pyrimidine rings of the nucleic acid strands and allows an electroanalytically usable redox reaction on working electrodes, is preferably an osmium (VIII) complex, preferably [OsO₄(bipy)] or [OsO₄(py)₂], in particular [OsO₄(bipy)]. Alternative redox markers are disclosed in WO 07/020093.

The nucleic acid in the sample may be RNA and/or DNA. DNA as well as RNA is generally at least partially double stranded, in particular at room temperature (approx. 20-25° C.). In the case of RNA this is due in particular to secondary structures.

The nucleic acid of the target strands can have a length of 100 to 5000 base pairs, preferably 500 to 2000 base pairs or be longer. In one embodiment, the nucleic acid in the sample is separated into shorter segments before the denaturation in step a), e.g. of 100 to 5000 base pairs, preferably 500 to 2000 base pairs. This can e.g. be achieved by treatment with nuclease or by ultrasound.

Preferably, the replacement of the protective strands by the immobilized probe strands in step d) occurs at a temperature that is optimal for the thermally stringent hybridization of probe and target strands. Preferably, the probe strands are immobilized on an electrode (working electrode) that is heated at the time of measurement and/or hybridization. That way, on the one hand the strand exchange at the surface of the electrode is accelerated and on the other hand the binding of strands that are not 100% homologous is made more difficult. The optimal temperature is below the so-called melting temperature. At the latter, 50% of both complementary strands are present as double strands. To determine the optimal temperature of each probe strand a melting curve analysis is used. For this, an analysis signal, which depends on the state of hybridization is plotted against the temperature. In a homogenous solution the UV-absorption or the fluorescence of the DNA will normally be measured to achieve this goal. If desired, several different probes complementary to the sequences to be detected may be used for detection of several sequences in a sample. Preferably, these different probes are immobilized on different selectively heated electrodes to set the respective optimal temperature during the hybridization and/or measurement.

In one embodiment, the surplus redox markers are removed from the analyte solution between step c) and d), e.g. by dialysis or filtration.

According to another variant of the method, the surplus redox markers remain in the the analyte solution after step c), i.e. the surplus redox markers are not removed between step c) and d) from the analyte solution, and the electrochemical signals of the redox markers bound to the surface of the electrode are separated from the signals of the redox marker in solution with suitable electrochemical analysis methods upon detection. According to this variant of the method chronocoulometry or chromopotentiometry as an electrochemical analysis method is preferred. The chronocoulometry allows the distinction between electrochemical diffusion currents which originate from the conversion of dissolved substances and electrochemical currents that are caused by substances fixed on surfaces. That way, for example, the amount of DNA immobilized on gold electrodes can be determined by addition of ruthenium hexamine chloride to the solution. This binds to the phosphate groups of the DNA strands (see, e.g., Steel, B. A. et al. Anal. Chem. 1998, 70, 4670-4677).

According to one embodiment, the probe strands are present in hybridized form in step d), i.e. they are hybridized with one or more nucleic acid strands as protective probe strands. In this case, the labeled nucleic acid target strands are hybridized in step d) at the surface of an electrode with the probe strands immobilized thereon thereby replacing the protective strands and the protective probe strands.

The electroanalytical detection of the target strands, which are bound to the immobilized probe strands, is made preferably by means of chronopotentiometry, coulometry, amperometry or voltammetry, preferably by means of square-wave voltammetry (SWV), alternating current voltammetrie (ACV) or cyclic voltammetry (CV) in the context of the method according to the invention. As an electrode metal, carbon, polymer or semiconductor electrodes are used, preferably wire or layer electrodes made of gold, platinum, copper, bismuth, mercury, silver, lead, tin or alloys thereof. Preferably, electrodes are used, which can directly be heated by electric current or indirectly be heated by a resistance heater. Preferably, a heatable electrode is directly heatable and has a uniform surface temperature.

The electrode surfaces can be modified with structures in the nanometer or micrometer range in order to increase their surface area. These structures may be of the same material as the electrode itself. The immobilization of the probe strands at the surface of the electrodes takes place according to methods known in the art. According to the invention, it is preferred to chemisorb the probe strands on metal electrodes preferably by means of thiol groups (see, e.g., Steel, B. A.; Herne, T. M.; Tarlov, M. J. Anal. Chem. 1998, 70, 4670-4677).

The invention also provides a device for carrying out a method according to the invention, comprising a flow system in which the mentioned steps of the method can take place in succession, wherein the flow system has heatable sections for thermal denaturation as well as coolable sections for rapid cooling of the sample. In particular, the section for thermal denaturation is heatable or heated to 93-97° C., preferably to 95° C. In the flow system, protective strand is added to the sample directly after, i.e., the system is designed such that protective strand may be added, e.g. by a channel mouth or injection device. Also this section may still be heatable or heated to the temperature of thermal denaturation or a temperature slightly below. The flow system is designed in such a manner that the sample can then be fed directly into a section which is cooled to a temperature suitable for lowering the sample temperature to 0° C. or less, preferably to about −1° C. to about −5° C., −2° C. to −4° C. or about −2.5° C. To this end, the section can for example be coolable or cooled to −2.5° C. to −25° C. or about −5° C. to −19° C. The temperature in the sample should not fall below the freezing point of the sample, while it passes through this cooled section. Due to the rapid cooling of the sample the advantageous effects explained above come into effect upon binding of the protective strands.

In one embodiment the device comprises a sample comprising nucleic acids, and the above-mentioned heatable and coolable sections are heated and cooled, respectively, to the appropriate temperatures.

In the device according to the invention preferably an array of different selectively heatable working electrodes is used as an electrochemical detector for electroanalytical detection of the nucleic acid strands hybridized to the probe strands, wherein different probes are immobilized on the working electrodes (i.e., probes with different sequence).

As an amplification of the target sequences is not necessary according to this method, a sufficient number of copies of the target sequence should be present in the sample. This is, for example, to be expected in the case of mRNA, rRNA and in samples containing a great many cells. Of course, the method according to the invention may also be used with samples that were previously amplified, e.g. with PCR or SDA.

According to the present invention, a sample may in particular be a biological or clinical sample, such as an extract of cells of plant, animal or human origin which comprises nucleic acids. The invention may, for example, be used advantageously for the detection of nucleic acids of a pathogen in a clinical sample or for the detection of genetically modified DNA of known sequence in a plant or food sample.

The economic benefits of the invention are reflected, for example, in that the sequence-specific analysis of DNA and RNA is much easier, since no PCR is required. Electrochemical DNA chips may be used which provide directly readable digital data. By use of electrode arrays many different sequences can be detected in parallel. Expensive, especially fluorescence-labeled oligonucleotides are no longer necessary. Also in the case of gene expression analyses, the expensive and time-consuming PCR amplification can be omitted. In addition, a direct quantification is given because the electrochemical signals are directly proportional to the concentration of the target sequences due to the elimination of the PCR.

In particular, the following commercially interesting applications of the principle according to the invention are opening up:
1. Direct quantitative detection of a DNA sequence of interest in a large excess of unspecific DNA without PCR, for example for the quantitative analysis of genetically modified organisms.
2. Rapid, simple, inexpensive electrochemical analysis of mRNA strands from the gene expression, quantitative and without prior PCR.
3. Quantitative, rapid, simple, inexpensive electrochemical analysis of bacteria via their specific rRNA in clinical samples,
4. Rapid, easy, inexpensive electrochemical multiplex-detection of many nucleic acid sequences in a sample using electrode arrays without the necessity of a multiplex PCR afflicted with compromises,
5. Selective and sensitive detection of PCR products.

### LEGEND

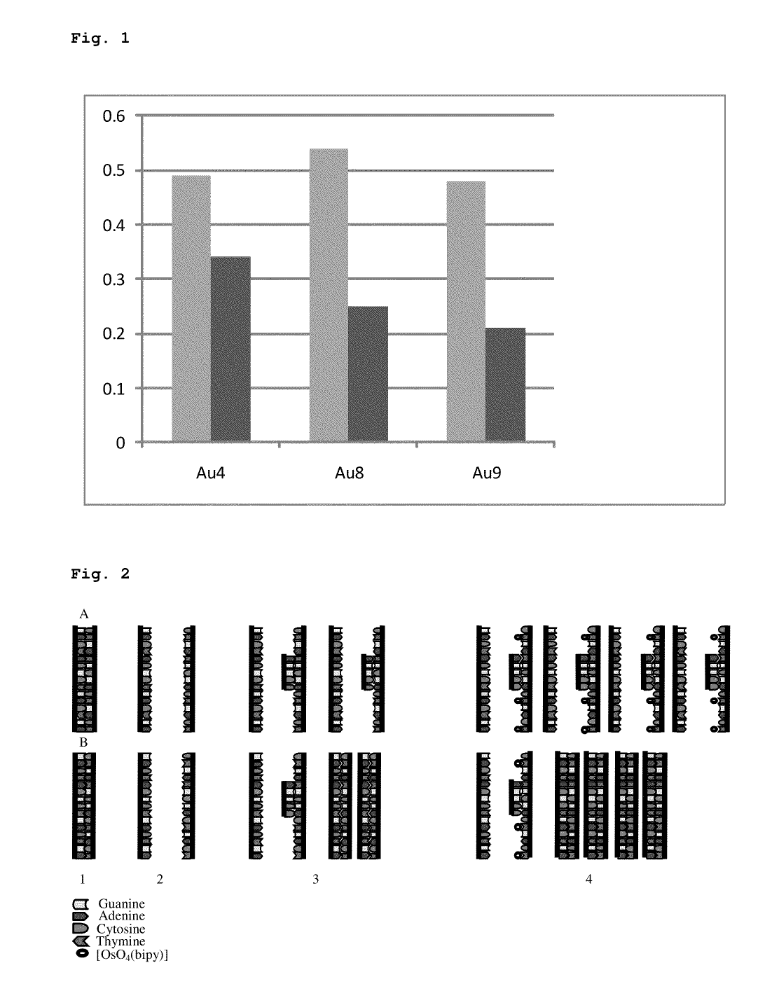

FIG. 1 shows the results of labeling PCR products with [OsO₄(bipy)] with different methods of hybridization with protective strand (see Example 1). Left bar: Method 1, right bar: Method 2. SWV signals in microampere.

FIG. 2 (A) shows the method for labeling PCR products according to the method 1, (B) shows the method according to method 2; 1. shows the double-stranded PCR products; 2. represents the strands after denaturation is; 3. shows the situation after the chilling, in method 1 the single strands remain separated from their complementary strands longer, so that the protective strands can hybridize more efficiently; 4. shows the situation after the labeling with (OsO₄[bipy]), there are significantly more labeled targets in method 1, which can hybridize with the probes on the electrode.

### EXAMPLES

### Example 1

### Labelling of Double Stranded PCR Products with [OsO₄(bipy)]

For labeling of PCR products with [OsO4 (bipy)] several identical PCR reactions were generated from a clinical sample, which contained Candida albicans DNA, and PCR was performed (Candida albicans: Primer: S1: 5′-ACTGCGAATGGCTCATTAAATCAG-3′ (SEQ ID NO:1), CUF1: 5′-CAAGGCCATGCGATTCG-3′ (SEQ ID NO:2); Probe: V2CA: 5′-TGCCTTCGGGCTCTTTGA-aaaaaaaaaaaaaaa[Dithio]₃-3′ (SEQ ID NO:3); protective strand: Schutz Canda: 5′-TACCTTCAGGCTCTTTAG-3′ (SEQ ID NO:4).

Hybridization with protective strands was performed differently according to the methods 1 (invention) and 2 (method modified according to the invention, by Th. Reske) as follows:

1. According to the invention, the sample was denatured in a thermocycler at 95° C. for 10 min. The vessel containing the reaction mixture was then transferred into a freezing mixture (ice bath with salt (NaCl)) and the reaction mixture was very rapidly cooled to −2.5° C. thereby. The protective strand was added and incubated for 10 min at this temperature. Then it was incubated for an additional maximal 110 min at room temperature.

2. According to the prior art (Reske, Ph.D. thesis 2009, p 18), the sample was only briefly denatured at 95° C. for max. 1 min. These attempts were not successful, i.e., only very small signals were obtained. Only through longer dwell times for denaturation and protective strand hybridization according to the invention useful, but relatively small signals were obtained at the end. In this example, therefore, denaturing has already been longer, i.e. according to the invention for minutes, than in the prior art. Subsequently, it was transferred to an ice bath (0-2° C.) and there the protective strand was added.

The reaction mixture was now cooled according to the invention for an extended period, i.e. for 10 min in the ice bath, then incubated for an additional maximal 110 min at room temperature.

Afterwards on all samples an addition of [OsO₄(bipy)] to a final concentration of 2 mM and further incubation for 2 h was performed.

The removal of [OsO₄(bipy)] was performed in both cases by overnight dialysis.

Results are shown in FIG. 1. The targets prepared according to the method of Thomas Reske modified according to the present invention (method 2) show significantly lower signals on three independently prepared electrodes (Au4, Au8, AU9).

Hybridization of the, thus, labeled targets to the probes was carried out at room temperature for 15 min. The measurement method used was the SWV (Square Wave Voltammetry): Scan −0.55 V-0 V, 200 Hz, step potential of 0.002 V, amplitude 0.04 V, used buffer: 10 mM Tris, 0.5 M Na₂SO₄, pH 7.5.

### Example 2

### Detection of 0.9% Traces of the MON810 Gene in the DNA of a Maize Sample by Labeling with [OsO₄(bipy)], Hybridization, and Electrochemical Analysis

A PCR reaction is performed with a template that contains 0.9% [w/w] of transgenic DNA (MON810 gene). In the PCR, a transgenic section is amplified. Subsequently, it is heated to 95° C. for 10 min to denature all double strands. Then the protective strand is added in excess. Then the sample is cooled according to the invention in a freezing mixture of ice/NaCl for 10 min to −2.5° C. Now the protective strand is attached to the matching sequences in the maize DNA in a thermally stringent manner. Subsequently, [OsO₄(bipy)] (preferably 2 mM) is added. After the reaction, the excess [OsO₄(bipy)] can be removed by dialysis. Finally, the hybridization with probe strands immobilized on gold is carried out under replacement of the protective strands and the electrochemical detection is carried out, for example, by SWV. The large excess of non-specific DNA and RNA was labeled in the labeling step with osmium tetroxide complex and is, since, no longer capable of hybridizing. Therefore, voltammetric signals of osmium tetroxide complex marker will only be obtained when the protective strand find its counterpart (MON810 section) in the amplified maize DNA and could hybridize therewith.

### Example 3

### Analysis of the Gene Expression by Labeling the mRNA Copies with [OsO₄(bipy)]

The nucleic acid extracts of a cell or tissue sample are denatured at 95° and the protective strands are added at the end of the denaturation, chilled by cooling the mixture to −2.5° C., and subsequently hybridized at room temperature with the protective strands. The labeling principle follows that one presented in Example 1. However, here it has to be made sure that care is taken regarding the absence of RNases until the labeling of the RNA. Then [OsO₄(bipy)] (preferably 2 mM) is added. Most of the nucleic acid strands (DNA and RNA) exists in single-stranded form and is modified with osmium tetroxide in the process and is no longer able to form double strands. Only the protected sections of the mRNA copies remain unlabeled and can, therefore, under replacement of the protective strands hybridize subsequently with the probe strands immobilized on gold. Thereby, a high selectivity on the one hand and a very high sensitivity on the other hand are achieved in the subsequent electrochemical analysis for example by chronopotentiometry. Reproduction of mRNA copies by RT-PCR and PCR can be omitted.

### Example 4

### Detection of Bacterial Cells by Labeling the Specific rRNA with [OsO₄(bipy)]

The nucleic acid extracts of a clinical sample are denatured at 95° and the protective strands are added, chilled very rapidly by means of a freezing mixture to −2.5° C. and subsequently hybridized with the protective strands at room temperature the protective strands. Then [OsO₄(bipy)] (preferably 2 mM) is added. Most of the nucleic acid strands (DNA and RNA) is present as single strand, is modified with osmium tetroxide bipyridine and is no longer able to form double strands. Only the protected sections of the rRNA copies can then hybridize with the probe strands immobilized on gold under replacement of the protective strands. Thereby, a high selectivity on the one hand and a very high sensitivity on the other hand are achieved in the subsequent electrochemical analysis for example by chronopotentiometry. A duplication of the rRNA copies by RT-PCR and PCR is no longer necessary. This principle can be used for identification and quantification of infectious bacteria in clinical samples. Also in this case it is essential that care is taken regarding the absence of RNases until the RNA is labeled with [OsO₄(bipy)].

### Example 5

### Electrochemical Analysis of Os-Labeled Nucleic Acids Directly After the Labeling Reaction without Prior Removal of the Excess [OsO₄(bipy)] in Solution

By applying the chronocoulometry one can differentiate between the electrochemical signals of the immobilized osmium compounds and those of the dissolved [OsO₄(bipy)] molecules. Therefore, removal of excess osmium complexes is not necessary. For implementation, the immobilized probes have to be protected with suitable protective probe strands. The latter are complementary to the immobilized probes, but preferably also contain 3 to 5, preferably 4 base mismatches to promote the hybridization of probe and target. The potential jump occurs from −500 to −200 mV (vs. SCE). For evaluation, the procedure is analogous to the method according to Steel et al. (Steel et al., Anal. Chem. 1998, 70, 4670-4677, FIG. 4), i.e. the chronocoulometric measurements are plotted against the square root of the time. The linear section is extrapolated to t=0, the intersection of this line with the charge axis gives the charge as measured value. From this, the blank value is still to be subtracted, which results from the capacitive charge reversal of the electrochemical double layer. The resulting charge is due to the electrochemical conversion of the markers immobilized on the probe layer with the target, and proportional to the target concentration.

### Example 6

### Electrochemical Analysis of the Os-Labeled Nucleic Acid Sequences by Thermally Stringent Hybridization on Selectively Heated Electrode Arrays

Each individual electrode is brought to a temperature in the analysis at which the capture probe immobilized thereon optimally and thermally stringent hybridizes to the target sequence. This further supports the specific detection of different target sequences in the presence of a large excess of non-specific nucleic acids. The electrode arrays, which can be used according to the invention, are, for example, described in the Ph.D. thesis of Duwensee H., 2009, or in DE 10 2004 017 750 B4.

### Example 7

### Electrochemical Analysis of Os-Labeled Sequences on Nanostructured Electrodes

The deposition of nanostructures made of gold and/or silver increases the active surface of the electrode and improves both the efficiency and stringency of hybridization, as well as the sensitivity and the signal-to-noise ratio. Therefore, the detection limit and the selectivity are further improved. Suitable nanostructures are described in Wachholz et al., Electroanalysis 21 (19) (2009) 2153 or French R et al. J Electroanal Chem 632 (2009) 206 and French R et al. Electroanalysis 20 (22) (2008) 2403. The deposition may be performed from both cyanide and other galvanic baths according to the state of the art.

Literature;

Foita et al., 1 Am. Chem. Soc. 126 (2004) 6532.

Kostecka et al., Bioelectrochemistry 63 (2004) 245.

Foita et al., Electroanalysis 15 (2003) 431.

Flechsig et al., Langmuir 21 (2005) 7848.

Flechsig et al., Anal. Chem 79 (2007) 2125.

French R et al., J Electroanal Chem 632 (2009) 206

French R et al., Electroanalysis 20 (22) (2008) 2403

Reske et al., Talanta 74 (2007) 393

Mix et al., Electroanalysis 21 (2009) 826

F. Wachholz et al., Electroanalysis 21 (2009) 2153

Steel et al., Anal. Chem. 1998, 70, 4670-4677

H. Duwensee, M. Mix, G.-U. Flechsig, Bioanalytical Reviews 1-4 (2010) 103

Reske, Ph.D. thesis, 2009

DE 10 2005 039 726

DE 10 2004 017 750 B4

WO 07/020093

1. A method for the sequence-specific detection of nucleic acids in a sample, in which the nucleic acids are at least partially present as double strands, comprising
a) converting the at least partially double stranded nucleic acid strands into single strands termed target strands by thermal denaturation,
b) adding at least one nucleic acid strand termed protective strand, which is able to hybridize with a target strand to form partially double stranded segments, wherein the protective strands are shorter than the target strands, wherein the temperature of the sample is rapidly lowered to a temperature of less than 5 ° C.
c) labeling the remaining single stranded segments of the target strands via reaction with a redox marker, which reacts selectively with the double bond of the pyrimidine rings of the nucleic acid strands and allows an electroanalytically usable redox reaction on working electrodes,
d) hybridizing the nucleic acid strands that are labeled in this manner on the surface of an electrode with probe strands that are immobilized thereon under replacement of the protective strands, and
e) detecting the nucleic acid strands that are hybridized to the probe strands electroanalytically.

2. The method according to claim 1, wherein the thermal denaturation in step a) takes place at at least 93° C. for more than 1 min.

3. The method according to claim lone of claim 1, wherein the addition of the protective strand in step b) takes place at a temperature of approximately the thermal denaturation temperature.

4. The method according to claim 1, wherein the temperature of the sample is rapidly lowered to a temperature of less than 0° C. by transferring the sample to an environment of less than 0° C. immediately after addition of the protective strand in step b).

5. The method according to claim 1, wherein the temperature of the sample in step b) is about −1.5° C. to −3° C., wherein this temperature is particularly achieved in a freezing mixture of ice with a salt.

6. The method according to claim 1, wherein the temperature in step b) is maintained for at least 1 min.

7. The method according to claim 1,
wherein the protective strand is half as long as the target strand at most,
wherein the protective strand is preferably designed in such a manner that one or more mismatches occur in the double stranded segments, and/or
wherein the protective strand is added in an excess of at least 2:1.

8. The method according to claim 1, wherein the redox marker that reacts selectively with the double bond of the pyrimidine rings of the nucleic acid strands and allows an electroanalytically usable redox reaction on working electrodes is an osmium (VIII) complex.

9. The method according to claim 1, wherein the nucleic acids in the sample are RNA and/or DNA.

10. The method according to claim 1, wherein the nucleic acids in the sample are separated into shorter segments before the denaturation in step a), wherein this can be achieved by treatment with nuclease or restriction endonuclease.

11. The method according to claim 1, wherein the replacement of the protective strands by the immobilized probe strands occurs at a temperature that is optimal for the thermally stringent hybridization of probe and target strands, wherein the probe strands are immobilized on a heatable electrode.

12. The method according to claim 1, wherein the surplus redox markers are removed from the analyte solution between step c) and d).

13. The method according to claim 1, wherein the surplus redox markers are not removed from the analyte solution between step c) and d), but remain in the sample, wherein the electrochemical signals of the osmium bound on a surface are separated from the signals of the osmium in solution during the detection by means of suitable electrochemical analysis methods.

14. A device for carrying out a method according to claim 1, comprising a flow system in which the method steps can take place in succession, wherein the flow system has heatable sections for thermal denaturation as well as coolable sections for rapid cooling of the sample.

15. The device according to claim 14, which comprises an array of different, selectively heatable working electrodes as an electrochemical detector for electroanalytical detection of the nucleic acid strands hybridized to the probe strands, wherein different probes are immobilized on the working electrodes.

16. The method of claim 1, wherein the thermal denaturation in step a) takes place at at least 95° C. for at least 5 min.

17. The method of claim 1, wherein the protective strand has a length of 10-200 b.

18. The method of claim 1, wherein the redox marker that reacts selectively with the double bond of the pyrimidine rings of the nucleic acid strands and allows an electroanalytically usable redox reaction on working electrodes is [OsO₄(bipy)] or [OsO₄(py)₂].

19. The method of claim 1, wherein the nucleic acids in the sample are separated into shorter segments of 100 to 5000 base pairs before the denaturation in step a).

20. The method of claim 1, wherein the replacement of the protective strands by the immobilized probe strands occurs at a temperature that is optimal for the thermally stringent hybridization of probe and target strands, wherein several different probes are used, which are immobilized on different, selectively heated electrodes, to set the optimal temperature for each probe sequence during the hybridization and/or measurement.


SEQUENCE LISTING (4 sequences)

SEQ ID NO: 1 - DNA, 24 bp, Artificial Sequence
  Primer S1
actgcgaatggctcattaaatcag

SEQ ID NO: 2 - DNA, 17 bp, Artificial Sequence
  Primer CUF1
caaggccatgcgattcg

SEQ ID NO: 3 - DNA, 33 bp, Artificial Sequence
  Sonde V2CA
tgccttcgggctctttgaaaaaaaaaaaaaaaa

SEQ ID NO: 4 - DNA, 18 bp, Artificial Sequence
  Schutz Canda
taccttcaggctctttag